(19) United States
(12) Patent Application Publication
(10) Pub. No.: US 2023/0372427 A1
(43) Pub. Date: Nov. 23, 2023

(54) THE COMPOSITIONS OF PLANT INGREDIENTS AND HERBS AGAINST CORONAVIRUS

(71) Applicant: LumiSTAR Biotechnology, Inc., Taipei City (TW)

(72) Inventors: Yu-Fen Chang, Taipei City (TW); Hsiu-Mei Wang, Taipei City (TW); Szu-Yu Lin, Taipei City (TW); Min-Wen Chung, Taipei City (TW)

(21) Appl. No.: 18/320,087

(22) Filed: May 18, 2023

Related U.S. Application Data
(60) Provisional application No. 63/343,586, filed on May 19, 2022.

(51) Int. Cl. A61K 36/82; A61K 36/185; A61K 36/46; A61K 36/53; A61K 36/232; A61K 36/48

(52) U.S. Cl. CPC A61K 36/82; A61K 36/185; A61K 36/46; A61K 36/53; A61K 36/232; A61K 36/48

(57) ABSTRACT

A composition of plant ingredients and a composition of herbs are introduced. The composition of plant ingredients and the composition of herbs respectively include Camellia, lotus seedpod of Nelumbo nucifera, Eucommia ulmoides Oliver, Glechoma hederacea and Angelica keiskei. The composition of plant ingredients and the composition of herbs can inhibit coronavirus infection in host cells.

### BACKGROUND

### 1. Technical Field

The present disclosure relates to a composition of plant ingredients, and particularly to a composition of plant ingredients for inhibiting coronavirus infection and a composition of herbs comprising those plant ingredients.

### 2. Description of the Related Art

Coronavirus is a ribonucleic acid (RNA) virus with an envelope which can infect mammals and birds. Currently known coronaviruses that can infect human cells include human coronavirus 229E (hereinafter referred to as HCoV-229E), severe acute respiratory syndrome coronavirus (hereinafter referred to as SARS-CoV), Middle East respiratory syndrome coronavirus (hereinafter referred to as MERS-CoV), and Severe Acute Respiratory Syndrome Coronavirus 2 (hereinafter referred to as SARS-CoV-2). Those coronaviruses may cause the common cold or cause serious illness.

Coronavirus Disease 2019 (hereinafter referred to as COVID-19) is acute pneumonia caused by SARS-CoV-2. The main transmission route of SARS-CoV-2 is droplet infection or contact infection, and SARS-CoV-2 has currently caused a worldwide pandemic. Common symptoms of patients with COVID-19 include fever, dry cough, fatigue, shortness of breath, muscle pain, headache, sore throat, and diarrhea. The current administration method for COVID-19 mainly relies on supportive therapy. It would be very helpful if there were other strategies for the prevention of COVID-19 infection effectively, especially through the utilization of natural resources such as plants or herbs.

### SUMMARY

In view of the aforesaid drawbacks of the prior art, the main object of the disclosure is to provide a composition of plant ingredients and a composition of herbs which have specific plant ingredients for inhibiting coronavirus infection.

To achieve the above object, the disclosure provides a composition of plant ingredients, which comprises Camellia, lotus seedpod of Nelumbo nucifera, Eucommia ulmoides Oliver, Glechoma hederacea, and Angelica keiskei.

According to an embodiment of the disclosure, the composition of plant ingredients comprises 3 parts by weight of Camellia, 3 parts by weight of lotus seedpod of Nelumbo nucifera, 2 parts by weight of Eucommia ulmoides Oliver, 3 parts by weight of Glechoma hederacea and 2 parts by weight of Angelica keiskei.

According to an embodiment of the disclosure, the composition of plant ingredients further comprises lotus seed of Nelumbo nucifera and Trigonella foenum-graecum.

According to an embodiment of the disclosure, the composition of plant ingredients comprises 3 parts by weight of Camellia, 3 parts by weight of lotus seedpod of Nelumbo nucifera, 2 parts by weight of Eucommia ulmoides Oliver, 3 parts by weight of Glechoma hederacea, 2 parts by weight of Angelica keiskei, 1 part by weight of lotus seed of Nelumbo nucifera and 1 part by weight of Trigonella foenum-graecum.

To achieve the above object, the disclosure further provides a composition of herbs for inhibiting coronavirus infection, which comprises Camellia, lotus seedpod of Nelumbo nucifera, Eucommia ulmoides Oliver, Glechoma hederacea and Angelica keiskei.

According to an embodiment of the disclosure, the composition of herbs further comprises 3 parts by weight of Camellia, 3 parts by weight of lotus seedpod of Nelumbo nucifera, 2 parts by weight of Eucommia ulmoides Oliver, 3 parts by weight of Glechoma hederacea and 2 parts by weight of Angelica keiskei.

According to an embodiment of the disclosure, the composition of herbs further comprises lotus seed of Nelumbo nucifera and Trigonella foenum-graecum.

According to an embodiment of the disclosure, the composition of herbs comprises 3 parts by weight of Camellia, 3 parts by weight of lotus seedpod of Nelumbo nucifera, 2 parts by weight of Eucommia ulmoides Oliver, 3 parts by weight of Glechoma hederacea, 2 parts by weight of Angelica keiskei, 1 part by weight of lotus seed of Nelumbo nucifera and 1 part by weight of Trigonella foenum-graecum.

According to an embodiment of the disclosure, the composition of herbs is used for blocking the entry of coronavirus, inhibiting syncytia formation or restricting forming area of the syncytia.

In continuation of the description above, the composition of plant ingredients or the composition of herbs, which comprise Camellia, lotus seedpod of Nelumbo nucifera, Eucommia ulmoides Oliver, Glechoma hederacea and Angelica keiskei, is used to inhibit coronavirus infection of host cells.

### BRIEF DESCRIPTION OF THE DRAWINGS

FIG. 1(A) and FIG. 1(B) show the results of the inhibitory effects on viral entry of wild-type SARS-CoV-2 pseudotyped lentivirus after treatment with a composition RXC19-A, a composition RXC19-B and the plant ingredients thereof according to an embodiment of the present disclosure;

FIG. 2 shows relation curves of the inhibitory effects of the plant ingredients LCV01 to LCV07, the composition RXC19-A, and the composition RXC19-B at different concentrations on blocking the infection and viral entry of SARS-CoV-2 pseudotyped lentivirus;

FIG. 3(A) shows fluorescence imaging of syncytia formation induced by the spike protein of wild-type SARS-CoV-2;

FIG. 3(B) shows bar charts of the number analysis of the syncytia formation shown in FIG. 3(A); and

FIG. 3(C) shows bar charts of the area analysis of the syncytia formation shown in FIG. 3(A).

### DETAILED DESCRIPTION OF THE EMBODIMENTS

The terms “an embodiment” and “in an embodiment” employed herein mean that the embodiment described may include a specific appearance, feature, structure or characteristic, but it is not limited that every embodiment must include the specific appearance, feature, structure or characteristic. In addition, the terms can but do not necessarily refer to the same embodiments mentioned in other parts of the specification. Moreover, when a specific module, appearance, feature, structure, or characteristic is described and combined into an embodiment, no matter whether there is a clear description in the specification, those skilled in the art can still combine the module, appearance, features, structures, or characteristics into other embodiments. In other words, any module, element, or feature can be combined with other elements or features in different embodiments unless it has obviously or inherently incompatible characteristics or is specifically excluded.

In this embodiment, two kinds of compositions of herbs which can effectively inhibit the infection and the entry of coronavirus into host cells are provided, and they are respectively referred to as RXC19-A and RXC19-B. First, the composition of herbs RXC19-A comprises the plant ingredients of Camellia, lotus seedpod of Nelumbo nucifera, Eucommia ulmoides Oliver, Glechoma hederacea and Angelica keiskei. The composition of herbs RXC19-B comprises the plant ingredients of Camellia, lotus seedpod of Nelumbo nucifera, Eucommia ulmoides Oliver, Glechoma hederacea, Angelica keiskei, lotus seed of Nelumbo nucifera and Trigonella foenum-graecum. In addition, the plant ingredients Camellia, lotus seedpod of Nelumbo nucifera, Eucommia ulmoides Oliver, Glechoma hederacea, Angelica keiskei, lotus seed of Nelumbo nucifera and Trigonella foenum-graecum can also be used as herbs. Thus, the compositions of herbs RXC19-A and RXC19-B are also the compositions of plant ingredients RXC19-A and RXC19-B mentioned in this disclosure and will be referred to as the compositions RXC19-A and RXC19-B hereafter. The preparation of the compositions RXC19-A and RXC19-B of this embodiment will be described below first, and the efficacy of the compositions RXC19-A and RXC19-B of this embodiment in inhibiting the infection and the viral entry into the host cells will be exemplified.

The Preparation of the Compositions of Herbs of this Embodiment:

First, multiple kinds of plant ingredients, comprising Camellia, lotus seedpod of Nelumbo nucifera, Eucommia ulmoides Oliver, Glechoma hederacea, Angelica keiskei, lotus seed of Nelumbo nucifera and Trigonella foenum-graecum, are prepared.

Then the above plant ingredients are dried to provide desiccated Camellia, lotus seedpod of Nelumbo nucifera, Eucommia ulmoides Oliver, Glechoma hederacea, Angelica keiskei, lotus seed of Nelumbo nucifera and Trigonella foenum-graecum. In this embodiment, a low-temperature drying process can be used. Finally, the above plant ingredients are mixed in the proportions of 3 parts by weight of Camellia, 3 parts by weight of lotus seedpod of Nelumbo nucifera, 2 parts by weight of Eucommia ulmoides Oliver, 3 parts by weight of Glechoma hederacea and 2 parts by weight of Angelica keiskei, for the composition of herbs RXC19-A of this embodiment, or in the proportions of 3 parts by weight of Camellia, 3 parts by weight of lotus seedpod of Nelumbo nucifera, 2 parts by weight of Eucommia ulmoides Oliver, 3 parts by weight of Glechoma hederacea, 2 parts by weight of Angelica keiskei, 1 part by weight of lotus seed of Nelumbo nucifera and 1 part by weight of Trigonella foenum-graecum for the composition of herbs RXC19-B of this embodiment.

Samples Preparation of Each of the Above Plant Ingredients:

The above dry plant ingredients (i.e., the desiccated Camellia, lotus seedpod of Nelumbo nucifera, Eucommia ulmoides Oliver, Glechoma hederacea, Angelica keiskei, lotus seed of Nelumbo nucifera and Trigonella foenum-graecum) are extracted using ethanol at room temperature for three times respectively, with each extraction lasting for three days. Those ethanol solutions are condensed under reduced pressure to yield ethanolic extracts, that is, the crude extracts. The dried crude extracts are dissolved in Dimethyl sulfoxide (DMSO) at a concentration of 100 mg/mL and sonicated for 30 minutes at 25° C. Then the unsolvable residuals are removed by centrifuge filtration, and the supernatant liquids are the samples of those plant ingredients. It should be noted that, in the following descriptions and in the Figures of the present disclosure, the Camellia sample is referred to as LCV01, the lotus seed of Nelumbo nucifera sample is referred to as LCV02, the lotus seedpod of Nelumbo nucifera sample is referred to as LCV03, the Eucommia ulmoides Oliver sample is referred to as LCV04, the Glechoma hederacea sample is referred to as LCV05, the Angelica keiskei sample is referred to as LCV06, and the Trigonella foenum-graecum sample is referred to as LCV07.

Furthermore, the composition RXC19-A is a mixture of the five plant ingredients LCV01 (i.e., Camellia), LCV03 (i.e., lotus seedpod of Nelumbo nucifera), LCV04 (i.e., Eucommia ulmoides Oliver), LCV05 (i.e., Glechoma hederacea) and LCV06 (i.e., Angelica keiskei) with a weight ratio of 3:3:2:3:2. The composition RXC19-B is a mixture of the seven plant ingredients LCV01 (i.e., Camellia), LCV03 (i.e., lotus seedpod of Nelumbo nucifera), LCV04 (i.e., Eucommia ulmoides Oliver), LCV05 (i.e., Glechoma hederacea), LCV06 (i.e., Angelica keiskei), LCV02 (i.e., lotus seed of Nelumbo nucifera) and LCV07 (i.e., Trigonella foenum-graecum) with a weight ratio of 3:3:2:3:2:1:1.

Example 1 of Pseudotyped Virus Neutralization Assay:

In this example, the pseudotyped virus neutralization assay was used to observe whether the plant ingredients LCV01 to LCV07 and the compositions RXC19-A and RXC19-B can effectively inhibit coronavirus infection of host cells by blocking viral entry. Specifically, a life cycle of SARS-CoV-2 includes entry to a host cell, replication of genomic ribonucleic acid (RNA), viral assembly and budding via exocytosis and release from the host cell. The entry of SARS-CoV-2 to the host cells exploits the spike protein on its viral envelope binding to the Angiotensin-converting enzyme 2 (hereinafter referred to as ACE2) receptor of the host cells.

Thus, the wild-type SARS-CoV-2 pseudotyped lentivirus expressing Green fluorescent protein (hereinafter referred to as WT-GFP-SARS-CoV-2-pseudotyped lentivirus) and the Human Embryonic Kidney Cells 293T (hereinafter referred to as 293T cells) expressing ACE2 are used in this example. The 293T cells expressing ACE2 were cultured in reduced serum mediums (hereinafter referred to as Opti-MEM) with the plant ingredients LCV01 to LCV07 and the compositions RXC19-A and RXC19-B in some specific concentrations first, followed by incubation with WT-GFP-SARS-CoV-2-pseudotyped lentivirus to observe whether the plant ingredients and the compositions could effectively inhibit the viral entry and infection of the 293T cells by the GFP-SARS-CoV-2-pseudotyped lentivirus or not.

Specifically, the 293T cells (1×10⁴ cells) stably expressing human ACE2 genes were seeded in each well of a black 96-well plate containing 50 μL of Opti-MEM and were cultured overnight at 37° C. under 5% CO₂. On the following day, the overnight medium in the 96-well plate was replaced with fresh Opti-MEM containing the plant ingredients LCV01 to LCV07 and the compositions RXC19-A and RXC19-B, with the final concentration of 10 μg/mL, respectively. The 293T cells were incubated with each of the plant ingredients LCV01 to LCV07 and the compositions RXC19-A and RXC19-B at 37° C. under 5% CO₂ for 1 hour, respectively. It should be noted that, Chloroquine (0.5 μg/mL), is used as a positive control for blocking viral entry. Solvent control was carried out by adding solvent vehicle only without containing any plant ingredients, compositions, or agents.

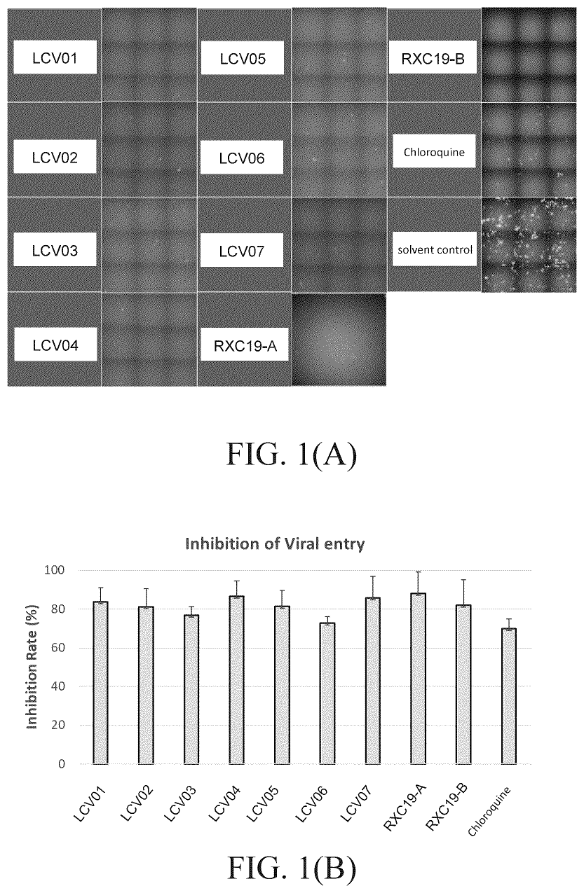

Then WT-GFP-SARS-CoV-2-pseudotyped lentivirus was transferred to the above 96-well plate containing 293T cells for transduction with the final multiplicity of infection (MOI) of 0.1. The culture medium was then replaced with fresh DMEM (supplemented with 10% FBS, 100 U/mL Penicillin and Streptomycin) at 16 hours post-infection and the 293T cells were continuously cultured for another 56 hours. After culturing the infected 293T cells at 37° C. under 5% CO₂ for 72 hours, the infected 293T cells were quantified for GFP fluorescence with an ImageXpress Micro Confocal High Content Imaging System. The experimental results are shown in FIG. 1(A) and FIG. 1(B). FIG. 1(A) and FIG. 1(B) show the results of the inhibitory effect on entry and infection of wild-type SARS-CoV-2 pseudotyped lentivirus after treatment with the compositions RXC19-A and RXC19-B and the plant ingredients thereof according to an embodiment of the present disclosure. Further, FIG. 1(A) presents fluorescence images, and FIG. 1(B) is a bar chart that quantifies the fluorescence and converts it into an inhibition rate of viral entry and infection.

In this example, the infected cells were stained with fluorescent dyes, DAPI, at 37° C. for 20 min, and then GFP-positive cell (i.e., the 293T cells successfully infected by the WT-GFP-SARS-CoV-2-pseudotyped lentivirus) and total cell nucleus were detected with an ImageXpress Micro Confocal High Content Imaging System. The total cells (indicated by nucleus staining) and GFP-positive cells were quantified for each well, and the transduction rates were determined by dividing the GFP-positive cell number to total cell number. Then the relative transduction rates were obtained by normalizing the transduction rates of plant ingredients, compositions, or chloroquine-treated groups to that of the solvent control (i.e., only solvent vehicles were added). Inhibition percentage was obtained by considering the solvent control as 0% inhibition, as shown in FIG. 1(B).

The pseudovirus used in this example, i.e., the WT-GFP-SARS-CoV-2-pseudotyped lentivirus, has a GFP gene; thus, after the entry to the host cells, GFP expression can be observed. In other words, successfully infected cells can be detected in green fluoresce, such as the fluorescence image of the non-treated solvent control (i.e., only solvent vehicle) shown in FIG. 1(A). In addition, FIG. 1(B) shows that the chloroquine (0.5 μg/mL)-treated positive control displayed about a 70% inhibitory effect. The rates of inhibition of viral entry of the plant ingredients LCV01 to LCV07 and the compositions RXC19-A and RXC19-B in 10 μg/mL are about 83%, 81%, 76%, 86%, 81%, 72%, 85%, 88% and 82%, respectively, as shown in FIG. 1(B). Thus, FIG. 1(A) and FIG. 1(B) show that the various plant ingredients (i.e., Camellia, lotus seedpod of Nelumbo nucifera, Eucommia ulmoides Oliver, Glechoma hederacea, Angelica keiskei, lotus seed of Nelumbo nucifera and Trigonella foenum-graecum) and the compositions thereof (i.e., RXC19-A and RXC19-B) used in this embodiment can effectively prevent the host cells from being infected by blocking viral entry.

Example 2 of Pseudotyped Virus Neutralization Assay:

In this example, the GFP-SARS-CoV-2-pseudotyped lentivirus were incubated in Opti-MEM with serial dilutions of the plant ingredients LCV01 to LCV07, as well as the compositions RXC19-A and RXC19-B, to pre-treat the SARS-CoV-2 pseudotyped lentivirus (Omicron type). Then the pseudovirus-plant ingredients mixture was added into 293T cells for transduction respectively to observe whether the plant ingredients and compositions could effectively inhibit the viral entry and infection or not in the same way as example 1. The half maximal inhibitory concentrations (IC₅₀) of plant ingredients LCV01 to LCV07 and compositions RXC19-A and RXC19-B against GFP-SARS-CoV-2 (Omicron type) were calculated.

The 293T cells (1×10⁴) stably expressing human ACE2 genes in 50 μL of Opti-MEM were seeded in each well of a black 96-well plate and cultured overnight at 37° C. under 5% CO₂. On the following day, plant ingredients LCV01 to LCV07 and compositions RXC19-A and RXC19-B were two-fold serially diluted in Opti-MEM, respectively, starting from 80 μg/mL. After six two-fold dilutions, a final concentration of 1.25 μg/mL can be obtained. The above plant ingredients LCV01 to LCV07 and compositions RXC19-A and RXC19-B serially diluted by Opti-MEM mediums were incubated with GFP-SARS-CoV-2-pseudotyped (Omicron type) lentivirus at 37° C. for 1 hour. Then the aforementioned mixtures (i.e., the mixtures of GFP-SARS-CoV-2-pseudotyped lentivirus (Omicron type) and each of the plant ingredients LCV01 to LCV07, and the mixtures of GFP-SARS-CoV-2-pseudotyped lentivirus (Omicron type) and each of the compositions RXC19-A and RXC19-B) were transferred to the plate seeded with 293T cells expressing ACE2 gene for transduction and with the final multiplicity of infection (MOI) of 0.1.

Similarly, the culture medium was replaced with fresh DMEM (supplemented with 10% FBS, 100 U/mL penicillin and streptomycin) at 16 hours post-infection, and the 293T cells were continuously cultured for another 56 hours. After culturing the infected 293T cells at 37° C. under 5% CO₂ for a total of 72 hours, 293T cells were quantified for GFP fluorescence with an ImageXpress Micro Confocal High Content Imaging System to calculate the half maximal inhibitory concentration (IC₅₀) of each of the plant ingredients LCV01 to LCV07 and each of the compositions RXC19-A and RXC19-B. The analysis of GFP fluorescence quantification (i.e., the calculations of the relative transduction rate and the inhibition rate) were performed as that in example 1 of the pseudotyped virus neutralization assay, and the details will not be described here.

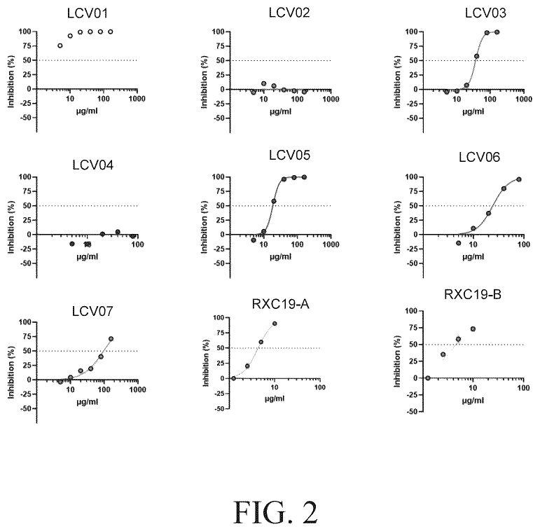

After calculations of the inhibition rate at each concentration of the plant ingredients LCV01 to LCV07 and compositions RXC19-A and RXC19-B against GFP-SARS-CoV-2-pseudotyped lentivirus (Omicron type), the relation curves of the inhibition rates versus concentrations of the plant ingredients or compositions were plotted by Prism 8 software (GraphPad), as shown in FIG. 2. FIG. 2 shows relation curves of the inhibitory effects of the plant ingredients LCV01 to LCV07, composition RXC19-A, and composition RXC19-B at different concentrations on blocking the infection and viral entry of SARS-CoV-2 pseudotyped lentivirus (Omicron type).

In this example, GFP-SARS-CoV-2-pseudotyped lentivirus (Omicron type) was pre-treated with the plant ingredients LCV01 to LCV07, as well as the compositions RXC19-A and RXC19-B, and then introduced to 293T cells for transduction to observe whether these plant ingredients and compositions could inhibit the infection and entry of GFP-SARS-CoV-2-pseudotyped lentivirus (Omicron type). As shown in FIG. 2, increases in the concentrations of the plant ingredients LCV01, LCV03, LCV05, LCV06, and LCV07, as well as the compositions RXC19-A and RXC19-B, resulted in decreased GFP expression in 293T cells (i.e., fewer 293T cells are infected by SARS-CoV-2 pseudotyped lentivirus (Omicron type)). The results suggest that the pre-treatment with the plant ingredients LCV01, LCV03, LCV05, LCV06 and LCV07, and the compositions RXC19-A and RXC19-B can inhibit the entry of SARS-CoV-2 pseudotyped lentivirus (Omicron type) into cells.

Next, a nonlinear regression method was used to determine the concentration at which 50% of GFP fluorescence (IC₅₀) was expressed. This concentration represents the half maximal inhibitory concentration (IC₅₀) of the ingredients (i.e., plant ingredients LCV01 to LCV07 and compositions RXC19-A and RXC19-B) for inhibiting SARS-CoV-2-pseudotyped lentivirus (Omicron type) entry, as shown in Table 1.

TABLE 1
The half maximal inhibitory concentration (IC₅₀) of plant ingredients
LCV01 to LCV07, as well as compositions RXC19-A and RXC19-B, for
inhibiting SARS-CoV-2-pseudotyped lentivirus (Omicron type) entry.

| | LCV01 | LCV02 | LCV03 | LCV04 | LCV05 | LCV06 | LCV07 | RXC19-A | RXC19-B |
|---|---|---|---|---|---|---|---|---|---|
| IC₅₀ | 2.94 | — | 37.17 | — | 18.70 | 24.25 | 95.69 | 4.29 | 4.40 |
| (μg/mL) | | | | | | | | | |

According to Table 1, plant ingredients LCV01 (i.e., Camellia), LCV03 (i.e., lotus seedpod of Nelumbo nucifera), LCV05 (i.e., Glechoma hederacea), and LCV06 (i.e., Angelica keiskei), and compositions RXC19-A and RXC19-B, have IC₅₀ values below 50 μg/mL and can effectively block the entry of SARS-CoV-2 lentivirus, thereby inhibiting the infection of cells. Among them, plant ingredient LCV01 (i.e., Camellia) and compositions RXC19-A and RXC19-B demonstrate better inhibitory effects.

Example 3 of Observing the Spike-Mediated Syncytia Formation:

This example was designed to observe whether the plant ingredients LCV01 to LCV07 and the compositions RXC19-A and RXC19-B have the ability to inhibit the spike-mediated syncytia formation. Specifically, when the spike protein on the SARS-CoV-2 envelope surface binds to the ACE2 receptor of the host cells, it can induce fusion between the bounded cell and neighboring host cells, resulting in the formation of syncytia that facilitate viral genome transfer to neighboring cells. In this example, two types of cells transfected with different vectors were prepared. One type of cell expressed the spike protein and the N-terminal constituents of Venus fluorescent protein, referred to as the effector cells. The other type of cell expressed the ACE2 and the C-terminal constituents of Venus fluorescent protein, referred to as the target cells. The target cells and the effector cells were co-cultured so that the spike protein on the effector cells would be able to bind to the ACE2 of the target cells, resulting in the formation of the syncytia. Concurrently, the N-terminal and C-terminal constituents of Venus fluorescent protein in the syncytia were combined to form a complete Venus fluorescent protein, which emitted green fluorescence signals for detection.

In this example, 293T cells expressing the spike protein and N-terminal components of Venus fluorescent protein (as effector cells) and 293T cells expressing ACE2 and C-terminal components of Venus fluorescent protein (as the target cells) were prepared separately. The target cells and the effector cells were pre-treated with 20 μM of the plant ingredients LCV01 to LCV07 and with compositions RXC19-A and RXC19-B at 37° C. under 5% CO₂ for 48 hours. In this example, there were two control groups; one was the “solvent control” group, and the other was the “negative control” group. The term“solvent control” refers to the pre-treatment of the target cells and effector cells were carried out by adding the solvent vehicles only, without the plant ingredients LCV01 to LCV07 or compositions RXC19-A and RXC19-B. As for the “negative control” group, spike and ACE2 proteins were not expressed in effector cells and target cells, respectively.

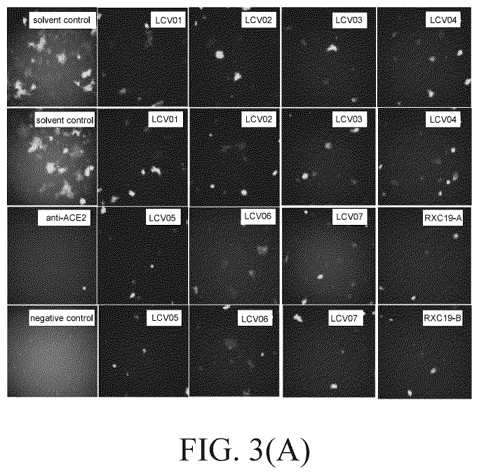

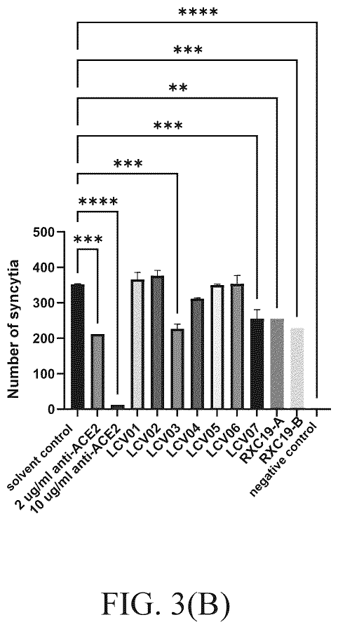

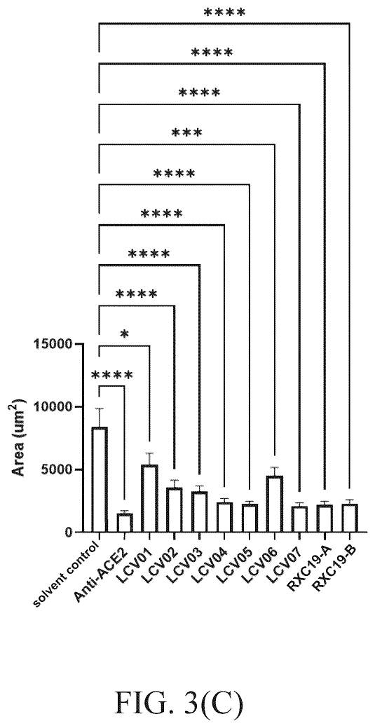

The pre-treated target cells and effector cells were then co-cultured for 5 hours. After 5 hours of incubation, syncytia (i.e., multinucleated cells) with expanded green fluorescence signals were detected and calculated to analyze the number and the area of syncytia formation, as shown in FIG. 3(A), FIG. 3(B) and FIG. 3(C). FIG. 3(A) shows fluorescence imaging results of syncytia formation induced by the spike protein of wild-type SARS-CoV-2; FIG. 3(B) shows a bar chart of the number analysis of the syncytia formation shown in FIG. 3(A); and FIG. 3(C) shows a bar chart of the area analysis of the syncytia formation shown in FIG. 3(A). In FIG. 3(B) and FIG. 3(C), the solvent control (i.e., solvent vehicle only) is used as a reference; a P value of less than 0.05 (i.e., p<0.05) is denoted with “*”; a P value of less than 0.01 (i.e., p<0.01) is denoted with “*”; a P value of less than 0.001 (i.e., p<0.001) is denoted with “***”; and a P value of less than 0.0001 (i.e., p<0.0001) is denoted with “****”.

Please refer to FIG. 3(A) and FIG. 3(B). Multinucleated cells with expanded green fluorescence signals formed in the solvent control group, which indicated spike-mediated syncytia formation. In contrast, in the negative control group without expressing spike and ACE2 proteins in 293T cells, no green fluorescence was detected, indicating syncytia were not able to be formed. The number of mediated syncytia formation was significantly reduced when cells were pre-treated with plant ingredients LCV03 (i.e., the lotus seedpod of Nelumbo nucifera), LCV07 (i.e., the Trigonella foenum-graecum), and compositions RXC19-A and RXC19-B, indicating that the plant ingredients LCV03 and LCV07 and the compositions RXC19-A and RXC19-B can inhibit syncytia formation effectively.

Please refer to FIG. 3(A) and FIG. 3(C). The area of syncytia formation in the solvent control group was larger than those in the experimental groups. After pre-treatment with plant ingredients LCV01 to LCV07 and compositions RXC19-A and RXC19-B, the area of syncytia formation were significantly reduced. This indicated that the plant ingredients LCV01 to LCV07 and compositions RXC19-A and RXC19-B can effectively restrict the forming area of syncytia, demonstrating their ability to suppress syncytia formation.

The above experimental results are summarized in Table 2. Specifically, the “Blocking viral entry” column in Table 2 was compiled based on the results in Table 1. Results for which the half-maximal inhibitory concentration (IC₅₀) could not be calculated or which showed an IC₅₀ greater than 50 μg/mL are marked as having no effect “−”. Results that showed an IC₅₀ greater than 10 μg/mL but less than or equal to 50 μg/mL are marked as having an effect “+”. Results that showed an IC₅₀ less than or equal to 10 μg/mL are marked as having a better effect “++”. Furthermore, the “Inhibit syncytia formation” column in Table 2 was compiled based on the results of the number analysis of syncytia formation in FIG. 3(B). Results that showed a significant difference are marked as having an effect “+”; results that did not show a significant difference are marked as having no effect “−”. The “Restrict forming area of syncytia” column in Table 2 was compiled based on the results of the area analysis of syncytia formation in FIG. 3(C). Results that showed a P value of less than 0.05 or less than 0.001 are marked as having an effect “+”, and results that showed a P value of less than 0.0001 are marked as having a better effect “++”.

TABLE 2
The inhibitory effect of the plant ingredients LCV01 to LCV07
and the compositions RXC19-A and RXC19-B on SARS-COV-2.

| | | | Restrict | Grading |
|---|---|---|---|---|
| | Blocking | Inhibit | forming | level of |
| | viral | syncytia | area of | inhibitory |
| | entry | formation | syncytia | effect |
| LCV01 | ++ | − | + | +++ |
| LCV02 | − | − | ++ | ++ |
| LCV03 | + | + | ++ | ++++ |
| LCV04 | − | − | ++ | ++ |
| LCV05 | + | − | ++ | +++ |
| LCV06 | + | − | + | ++ |
| LCV07 | − | + | ++ | +++ |
| RXC19- | ++ | + | ++ | +++++ |
| A | | | | |
| RXC19- | ++ | + | ++ | +++++ |
| B | | | | |

Table 2 shows that the plant ingredients LCV01 to LCV07 and the compositions RXC19-A and RXC19-B can inhibit SARS-CoV-2 viral infection through three different mechanisms (i.e., the middle three columns in Table 2). Specifically, the plant ingredients LCV01 (i.e., Camellia), LCV03 (i.e., lotus seedpod of Nelumbo nucifera), LCV05 (i.e., Glechoma hederacea) and LCV06 (i.e., Angelica keiskei) and the compositions RXC19-A and RXC19-B can effectively block viral entry and inhibit the infection of cells by SARS-CoV-2 pseudovirus. Among them, the plant ingredients LCV01 (i.e., Camellia) and the compositions RXC19-A and RXC19-B show even better inhibitory effects. The plant ingredients LCV03 (i.e., lotus seedpod of Nelumbo nucifera), LCV07 (i.e., Trigonella foenum-graecum) and the compositions RXC19-A and RXC19-B can effectively inhibit syncytia formation. In addition, the plant ingredients LCV01 to LCV07 and the compositions RXC19-A and RXC19-B can restrict the forming area of syncytia significantly, thereby effectively suppressing viral genome propagation. The effects of the three mechanisms are consolidated into the “Grading level of inhibitory effect” column in Table 2, which is the sum of the “+” symbols of the three columns to the left. According to the “Grading level of inhibitory effect” column in Table 2, the compositions RXC19-A and RXC19-B have better inhibitory effects on SARS-CoV-2 viral infections than those of the single plant ingredients. In other words, compared to using a single plant ingredient (LCV01 to LCV07), the compositions RXC19-A and RXC19-B are shown to have the highest levels of preventing viral infection. The compositions RXC19-A and RXC19-B can effectively block viral entry, inhibit the syncytia formation, and restrict the forming area of syncytia.

The above examples have exemplified that the components of RXC19-A and RXC19-B and the composition of herbs RXC19-A and RXC19-B have efficacy in inhibiting the infection of host cells by SARS-CoV-2 pseudovirus. In another embodiment, the plant ingredients and composition of herbs can be prepared using different ratios of Camellia, lotus seedpod of Nelumbo nucifera, Eucommia ulmoides Oliver, Glechoma hederacea, and Angelica keiskei, instead of using the ratios of RXC19-A or RXC19-B, and are applied to diseases caused by coronaviruses.

As described above, the composition of plant ingredients or the composition of herbs, which comprises Camellia, lotus seedpod of Nelumbo nucifera, Eucommia ulmoides Oliver, Glechoma hederacea and Angelica keiskei, can be used to inhibit coronavirus infection of host cells.

Although the present disclosure has been explained in relation to its preferred embodiments, it is to be understood that many other possible modifications and variations can be made without departing from the spirit and scope of the disclosure as hereinafter claimed.

What is claimed is:

1. A composition of plant ingredients, comprising Camellia, lotus seedpod of Nelumbo nucifera, Eucommia ulmoides Oliver, Glechoma hederacea and Angelica keiskei.

2. The composition of plant ingredients as claimed in claim 1, comprising 3 parts by weight of Camellia, 3 parts by weight of lotus seedpod of Nelumbo nucifera, 2 parts by weight of Eucommia ulmoides Oliver, 3 parts by weight of Glechoma hederacea and 2 parts by weight of Angelica keiskei.

3. The composition of plant ingredients as claimed in claim 1, further comprising lotus seed of Nelumbo nucifera and Trigonella foenum-graecum.

4. The composition of plant ingredients as claimed in claim 3, comprising 3 parts by weight of Camellia, 3 parts by weight of lotus seedpod of Nelumbo nucifera, 2 parts by weight of Eucommia ulmoides Oliver, 3 parts by weight of Glechoma hederacea, 2 parts by weight of Angelica keiskei, 1 part by weight of lotus seed of Nelumbo nucifera and 1 part by weight of Trigonella foenum-graecum.

5. A composition of herbs, comprising Camellia, lotus seedpod of Nelumbo nucifera, Eucommia ulmoides Oliver, Glechoma hederacea and Angelica keiskei.

6. The composition of herbs as claimed in claim 5, comprising 3 parts by weight of Camellia, 3 parts by weight of lotus seedpod of Nelumbo nucifera, 2 parts by weight of Eucommia ulmoides Oliver, 3 parts by weight of Glechoma hederacea and 2 parts by weight of Angelica keiskei.

7. The composition of herbs as claimed in claim 5, further comprising lotus seed of Nelumbo nucifera and Trigonella foenum-graecum.

8. The composition of herbs as claimed in claim 7, comprising 3 parts by weight of Camellia, 3 parts by weight of lotus seedpod of Nelumbo nucifera, 2 parts by weight of Eucommia ulmoides Oliver, 3 parts by weight of Glechoma hederacea, 2 parts by weight of Angelica keiskei, 1 part by weight of lotus seed of Nelumbo nucifera and 1 part by weight of Trigonella foenum-graecum.

9. A composition of herbs for inhibiting coronavirus infection, further comprising Camellia, lotus seedpod of Nelumbo nucifera, Eucommia ulmoides Oliver, Glechoma hederacea and Angelica keiskei.

10. The composition of herbs for inhibiting coronavirus infection as claimed in claim 9, comprising 3 parts by weight of Camellia, 3 parts by weight of lotus seedpod of Nelumbo nucifera, 2 parts by weight of Eucommia ulmoides Oliver, 3 parts by weight of Glechoma hederacea and 2 parts by weight of Angelica keiskei.

11. The composition of herbs for inhibiting coronavirus infection as claimed in claim 9, further comprising lotus seed of Nelumbo nucifera and Trigonella foenum-graecum.

12. The composition of herbs for inhibiting coronavirus infection as claimed in claim 11, comprising 3 parts by weight of Camellia, 3 parts by weight of lotus seedpod of Nelumbo nucifera, 2 parts by weight of Eucommia ulmoides Oliver, 3 parts by weight of Glechoma hederacea, 2 parts by weight of Angelica keiskei, 1 part by weight of lotus seed of Nelumbo nucifera and 1 part by weight of Trigonella foenum-graecum.

13. The composition of herbs for inhibiting coronavirus infection as claimed in claim 9, wherein the composition of herbs is used for blocking coronavirus entry, inhibiting syncytia formation, or restricting the forming area of syncytia.
